(19) United States
(12) Patent Application Publication
(10) Pub. No.: US 2012/0308625 A1
(43) Pub. Date: Dec. 6, 2012

(54) A METHOD FOR ALTERING THE FLAVOR OF A FOOD PRODUCT

(75) Inventor: Bernard Lahousse, Kortrijk (BE)

(73) Assignee: SENSE FOR TASTE, Brugge (BE)

(21) Appl. No.: 13/577,302

(22) PCT Filed: Feb. 7, 2011
(86) PCT No.: PCT/EP2011/051722

§ 371 Date: Aug. 6, 2012

(30) Foreign Application Priority Data
Feb. 5, 2010 (EP) 10152841.2

(51) Int. Cl. A23L 1/22; A23L 1/318; A23L 1/33; A23L 1/39; C12G 1/00; A61P 3/02; A23C 9/156; A23G 1/48; C12G 3/06; A61K 9/00; A61K 8/02; A23L 1/333; A23L 1/212

(52) U.S. Cl. 424/401; 426/281; 426/589; 426/592; 426/584; 426/631; 424/400

(57) ABSTRACT

A method for altering the flavor of a food product by impregnating a substance into a food product by exposing the food product to a pressure from 1000 to 6000 bar together with a solid compound containing the substance. The method further includes extracting the substance from the solid compound simultaneously with the step of impregnating.

### FIELD OF THE INVENTION

The present invention relates to a method for altering the flavor of a food product comprising impregnating a substance into a food product by exposing the food product to a pressure from 1000 to 6000 bar together with a compound containing the substance.

### BACKGROUND OF THE INVENTION

Impregnating or marinating of food in wine, spices, etc. at room temperature or below has been known for centuries. One huge disadvantage of this classical method of impregnation is the fact that this process is very slow, and thus very time-consuming. For example, in order to sufficiently impregnate meat with a specific flavor, it must be marinated for 2 to 3 days. Otherwise, the flavor isn't sufficiently absorbed into the meat.

Other examples of classical impregnation of food products are the pickling of meat or fish or the candying of fruits. Similar to marinating, these methods are also very slow and time-consuming.

More recently, impregnation of flavoring substances in food products such as meat, fish, has been carried out by vacuum techniques, wherein a food product is placed in a vacuum for a certain period of time, together with a liquid. For example, an apple placed in an amount of red beetroot juice under vacuum pressure, will result in a red apple that is completely impregnated with the flavor of the red beetroot. Several examples of this technique are described by Frito et al, 1994 (Vacuum osmotic dehydratation, Process optimization and minimal processing of foods).

However, a major disadvantage of this technique is that it can not be applied on all food products, and its efficiency is variable and very much depending of the specific application in which it is used. For example, shellfish cannot be impregnated with this technique because the shellfish meat cannot withstand a vacuum and will be destroyed.

Another impregnating technique is injecting substance with needles. For example, brine is often injected in meat as described in U.S. Pat. No. 5,934,187, or if applied on a larger, more industrial scale in WO2008135610.

A disadvantage of this technique however is the non-equal spreading of the fluid containing a certain aroma or flavor.

Another disadvantage is the fact that this technique can not be applied to food products suffering from increased fluid loss after injecting, such as for example shellfish.

Still another disadvantage is that, to allow injecting a fluid containing a substance, such fluid has to be prepared first, resulting in additional process steps, increased process time and increased production cost.

Other examples of impregnating food products are described in US2002/0009534 and in EP0719502. In US2002/0009534 shellfish is immersed in a pressurized fluid wherein substances like salt, lemon juice, hot sauces are added. In EP0719502, fruit juice is flavored by adding to the pressurized fluid a substance, either synthesized from natural products, either synthetic.

A disadvantage of the above methods is again that the substances have to be prepared first (by refinement, squeezing, chemical synthesis, etc.) and added to the pressurized fluid, resulting in additional process steps, increased process time and increased production cost.

Considering the above drawbacks, it is an object of the present invention to provide a method for altering the flavor of a food product, which is less suffering from variable efficiency, less suffering from application dependent performance, less suffering from unequal spreading of the aroma or flavor, and applicable to all food products.

It is an object of the present invention to provide a method for altering the flavor of a food product wherein the user is not forced to first prepare substances to be impregnated in the food product.

Another object of the present invention is to provide a method for altering the flavor of a food product wherein the user is not forced to use synthesized substances, or synthetic aromatizing substances.

It is another object of the present invention to provide a method having the ability to not merely flavoring the food product, but rather clearly alter the flavor of the food product.

Another object of the present invention is to provide a method allowing selecting the flavor to which the original flavor should alter.

The present invention addresses the above objects by impregnating a substance into the food product by means of exposing the food product to a pressure from 1000 to 6000 bar together with a solid compound containing the substance, wherein during exposure the substance is extracted from the solid compound simultaneously with the step of impregnating.

### SUMMARY OF THE INVENTION

The present invention is directed to a method for altering the flavor of a food product comprising impregnating a substance into a food product by exposing the food product to a pressure from 1000 to 6000 bar together with a compound containing the substance, characterized in that the compound is a solid compound and that the method further comprises extracting the substance from the solid compound simultaneously with the step of impregnating.

### DESCRIPTION OF THE INVENTION

In the context of the present invention, altering the flavor of a food product is understood as comprising not only merely flavoring the food product, but optionally even changing the original flavor of the food product into the flavor of the solid compound or of the substance extracted thereof.

In the context of the present invention, a substance is understood as a substance chosen from the group of aromas, aromatic molecules, colorings agents, softening agents, enzymes, gelating agents food additives, taste molecules, health improving components, texture changing components, vitamins, minerals, antimicrobial substances, and combinations of two or more thereof.

In the context of the present invention, a solid compound is understood as a solid food product, such as vegetables, fruit, meat, fish, shell fish, crustacean, nuts, cheese, cereals, spices, herbs, or as a non-food product such as wood, plants, or flowers.

According to a first embodiment of the present invention a method for altering the flavor of a food product is provided comprising impregnating a substance into a food product by exposing the food product to a pressure from 1000 to 6000 bar together with a compound containing the substance,

characterized in that the compound is a solid compound and that the method further comprises extracting the substance from the solid compound simultaneously with the step of impregnating.

The inventors have surprisingly found that it is possible to alter the flavor of a food product in a very short time span by simultaneously extracting a substance from a solid compound and impregnating that substance into a food product at a pressure from 1000 to 6000 bar.

A first advantage of such method is that it may be less suffering from variable efficiency, less suffering from application dependent performance, less suffering from unequal spreading of the aroma or flavor, and applicable to all food products.

Another advantage of such method may be that the user is not forced to first prepare substances to be impregnated in the food product. Moreover, he is not forced to use synthesized substances, or synthetic aromatizing substances. On the contrary, he may be directly use raw, natural, even completely untreated solid compounds containing the substance to be impregnated. For example, to impregnate ethyl dodecanoate in a food product, he may use natural raw coconut, or to impregnate beta-damascenone in a food product, he may use a rose.

A further advantage of such method may be that it has the ability to not merely flavoring the food product, but optionally clearly alter the flavor of the food product.

In embodiment in accordance with the present invention, the food product may be a solid or a liquid.

Such liquid food product may for example be water, bouillon, fumet, vegetable juices, fruit juices, alcoholic drinks, seawater, sugar syrop, oil, molten chocolate, water extracts from cheese, condiments, fond, alcoholic drinks, spirits, liquors, beer, wine, soft drinks, or energy drinks.

Such solid food product may be shell-fish, fish, meat, vegetables, fruit, nuts, cereals, eggs, fungi, mushrooms, coffee, dairy, plants, herbs, spices, seaweeds, seeds, flowers and derivates thereof and combinations thereof.

A preferred application of a method in accordance with the present invention may be altering the flavor of shell-fish, in particular bivalves. Since the meat of bivalves such as oysters and mussels is completely shut off from the outside world, it is very difficult to impregnate them with substances. Often, the only way to transfer some taste to a bivalve is to open them, marinate them or to process them at elevated temperature with a sauce. By using a method according to the present invention, shell fish such as bivalves are impregnated with substances without opening them before exposure to high pressure.

The food products may also be food supplements or vitamin supplements, or food products used in therapeutic or cosmetic compositions.

The high pressure used in a method in accordance with the present invention may be between 1000-6000 bar, preferably between 2000-6000, more preferably between 3000-6000 bar, most preferably between 4000-6000 bar.

The food product together with the solid compound may be exposed to a high pressure for a period of at least one second, preferably at least one minute, more preferably at least 3 minutes, but preferably no more than 5 minutes.

For very sensible food products, a time period of 1 second may be applied in order not to damage the food product. Generally speaking, 1 minute will be sufficient for a food product to absorb a substantial part of the substances from the solid compound. Above 5 minutes, the amount of substances absorbed by the food product may reduce drastically such that no additional benefits are gained from applying longer exposure times.

As a consequence, a product with a low shelf life, such as fresh shell-fish, can also be impregnated given the fact that the time to impregnate may be reduced from a couple of days to a couple of minutes, or even seconds.

In an embodiment in accordance with the present invention, a method for altering the flavor of a food product is provided, further comprising selecting the substance to be extracted from the solid compound, and determining the high pressure suitable for extracting the selected substance.

It is found by the inventors that the pressure used may have an influence on either which and which amount of substance is extracted from the solid compound, either which and which amount of substance is impregnated into the food product. For example, upon increasing the pressure when using star anise as solid compound, the food product is flavored with increasing black tea flavor.

It appears that seeking for an optimum between maximum flavor transfer from solid compound to food product, and the amount of tannins, or bitter or sour substances transferred results in an optimum pressure. For example, in case the solid compound is mushroom, ginger, pineapple, the optimum pressure is about 4000 bar, whereas for coriander, star anise, orange, natural vanilla, the optimum pressure is about 5000 bar, and for celery about 3000 bar.

The food product may be packed together with the solid compound in a packaging, preferably a vacuum packaging before exposure to high pressure, enhancing substance transfer between solid compound and food product.

Preferably, in case the food product is a liquid, the solid compound is at least partially immersed in the liquid. In case the food product is a solid, preferably both solid compound and food product are at least partially immersed in a liquid.

Preferably, the solid compound and the solid food product are in close proximity, by as understood by a person skilled in the art, a method in accordance with the present invention may be thought of wherein the solid compound and the solid food product are pressurized together, each in its own process cavity but both in contact with the liquid in which they are at least partially immersed.

In an embodiment in accordance with the present invention, a method for altering the flavor of a food product may be provided, further comprising selecting the substance to be extracted from the solid compound, and at least partially immersing the solid compound in a liquid selected for extracting the selected substance.

Such liquid selected for extracting the selected substance and in which the solid compound may be partially immersed may be either the food product itself, either a liquid used, without being bound by any theory, as a solvent for the selected substance. The choice of liquid may have an influence on either which and which amount of substance is extracted from the solid compound, either which and which amount of substance is impregnated into the food product. For example, upon using star anise as solid compound, the food product is flavored with respectively more anise flavor, or more black tea flavor by using alcohol in stead of water. It appears that seeking for an optimum between maximum flavor transfer from solid compound to food product, and the amount of tannins transferred results in an optimum pressure.

Liquids selected for extracting the selected substance may be water, bouillon, alcohol, or any liquid mentioned earlier.

A method in accordance with the present invention allows the building-up of a taste profile of a desired final product. By using the correct solid compound and the appropriate pressures and liquids to immerse the solid compound in, and by making suitable combinations or sequences thereof, the desired taste profile may be reached.

A method in accordance with the present invention may be used in a sequence of impregnating steps. For example, an orange as solid compound may be pressurized in water at 5000 bar, and subsequently after removing the orange, cockles may be impregnated with the obtained orange flavored water at 6000 bar.

A method in accordance with the present invention may additionally be used also for reducing or eliminating pathogenic organisms in the food product, particular Vibrio pathogens in oysters.

Such method may also additionally used for adjusting or improving the texture with respect to bite and volume of the food product.

Such method may also additionally be used to impregnate anti-microbial and/or therapeutic substances into liquid food products, or even directly into solid food products. For example, using a combination of star anise and elderberry as solid compound may result in impregnation of shikimic acid and flavonoides, which are substances known to have therapeutic power, for example in case of influenza.

Such method may also additionally used for adjusting or improving characteristics such as shelf live, color stability, or oxidation speed of food products by using solid compounds containing anti-oxidation agents, anti-coloring agents, etc.

Different experiments have been conducted on a high pressure device from NC Hyperbaric with a 55 liter content per batch, at a temperature of 5-30° C., with a maximum pressure of 600 MPa (=6000 bar). Examples are given below.

### Example 1

It is possible to transfer the taste of ginger to an oyster by simple placing a piece of ginger near the shell of a fresh closed oyster in water at 5000 bar for 1 minute.

Result: The taste of the ginger will completely be absorbed in the oyster without the oyster losing its natural flavor.

### Example 2

A fresh oyster was packed vacuum together with a piece of kiwi in water. The oyster was kept at 6000 bar for 1 minute.

Result: the oyster has a soft taste of kiwi combined with the natural taste of a fresh oyster. The texture has slightly evolved because of the high pressure so that an oyster was obtained with a little more bite,

### Example 3

A piece of lamb meat of 100 g was packed together with 5 grams of seaweed and 20 grams of bouillon. The meat was kept at 6000 bar for one minute.

Result: the meat is fully impregnated with the taste of the seaweed.

### Example 4

Raw cucumber and cockles are immersed in water for 1 minute at 5000 bar.

Result: the cockles have a strong cucumber taste which is surprising because naturally, raw cucumber tastes very weak.

### Example 5

Water was packed together with seaweed and a piece of lamb meat. This mixture was treated with a pressure of 6000 bar for five minutes.

Result: the water tasted like lamb and seaweed. This liquid could be used as a basis for sauces.

### Example 6

Wine was packed together with lemongrass. The mixture was treated with a pressure of 6000 bar for five minutes.

Result: the wine had the aromas of lemongrass.

### Example 7

Fresh grapes were packed together with white wine, apricot, cinnamon, roses. The mixture was treated at 6000 bar for 30 seconds.

Result: the grapes stayed crispy, gained in volume and the taste was clearly still grape, but enriched with rose, cinnamon, apricot flavors and wine.

### Example 8

Wine was packed with lemongrass, thyme, apricot and pressurized for 5 minutes at 6000 bar. The wine was enriched with all the flavors, but was also thickened by components from the apricot, more like a dessert wine.

### Example 9

Celery stalks were added to a bag filled with water and kiwi fruit. By applying 6000 bar during 4 minutes, the stalks gained in crispness and tasted like celery with small hints of kiwi.

### Example 10

Cucumber was packed with vodka and treated for 4 minutes at 6000 bar. The vodka obtained a very rich cucumber taste and flavor.

### Example 11

Strawberries were combined with milk and pressurized 4 minutes at 4000 bar. The colour from the strawberry together with the flavor were extracted into the milk. The colour and the flavor were very intense. The strawberry lost partially its colour and most of its flavor.

### Example 12

Fresh bananas were combined with molten chocolate. They were processed warm at 6000 bar during 5 minutes. The chocolate contains very distinct banana flavors after the process and after the bananas were removed from the chocolate.

### Example 13

Fresh cherries were packed with star anise, orange peel and vodka, and pressurized for 4 minutes at 5000 bar.

Result: cherries infused with alcohol, orange flavor and anise flavor.

### Example 14

Cherry tomatoes were combined with cucumber, orange peel, waste material of shrimps and salted water and pressurized for 4 minutes at 6000 bar. The cherry tomatoes contained the flavors, especially the peel of the tomato.

### Example 15

In whiskey, the following aroma's can be distinguished.
3-methylbutanal: malt
Vanillin: vanilla
Guaiacol: coffee
Maltol: larix wood
Eugenol: cloves
Oak lactone: licorice
Acetaldehyde: orange peel
Furaneol: Tamarinde
Furfural: pineapple
Ethyl octanoate: plum
Ethyl hexanoate: apple
4-vinylguaiacol: lovage root
2,3-butanedione: strawberry
Ethyl dodecanoate: coconut
Ethyl decanoate: plum
2-phenylethanol: grapes
Isoamyl acetate: banana
Beta-damascenone: rose

By using the corresponding natural solid compound mentioned next to each aroma, and using the appropriate pressures and liquids to immerse the solid compound in, and by making suitable combinations or suitable sequences thereof, a taste profile of whiskey may be obtained, which may be used in improving the quality of lower quality whiskeys.

Likewise applications may be in using wood, vanillin, etc. for improving the taste of wines, improving the taste of balsamic vinegar, improving the taste of gin, etc.

1. A method for altering the flavor of a food product comprising impregnating a substance into a food product by exposing the food product to a pressure from 1000 to 6000 bar together with a compound containing the substance, wherein the compound is a solid compound and that the method further comprises extracting the substance from the solid compound simultaneously with the step of impregnating.

2. The method according to claim 1, wherein the food product is a solid or a liquid.

3. The method according to claim 2 wherein the food product is chosen from a group comprising shell-fish, fish, meat, vegetables, fruit, nuts, cereals, eggs, fungi, mushrooms, coffee, dairy, plants, herbs, spices, seaweeds, seeds, flowers and derivates thereof and combinations thereof, or from a group comprising water, bouillon, fumet, vegetable juices, fruit juices, alcoholic drinks, seawater, sugar syrop, oil, molten chocolate, water extracts from cheese, condiments, fond, alcoholic drinks, spirits, liquors, beer, wine, soft drinks, or energy drinks

4. The method according to claim 3 wherein the food product is chosen from the group of mollusks or bivalves.

5. The method according to claim 1, that wherein the solid compound is chosen from the group comprising food products, vegetables, fruit, meat, fish, shell fish, crustacean, nuts, cheese, cereals, spices, herbs, plants, wood, flowers, or a combination thereof.

6. The method according to claim 1, wherein the substance is chosen from the group of aromas, aromatic molecules, colorings agents, food additives, taste molecules, health improving components, texture changing components, vitamins, minerals and combinations of two or more thereof.

7. The method according to claim 1, wherein the high pressure is between 4000-6000 bar.

8. The method according to claim 1, wherein the food product, together with the solid compound is exposed to a high pressure for a period from 1 second up to 5 minutes.

9. The method according to claim 1, further comprising selecting the substance to be extracted from the solid compound, and determining the high pressure suitable for extracting the selected substance.

10. The method according to claim1, further comprising selecting the substance to be extracted from the solid compound, and at least partially immersing the solid compound in a liquid selected for extracting the selected substance.

11. The method according to claim 1, wherein the food product is packed together with the solid compound in a packaging.

12. The method according to claim 11, wherein the food product is packed together with the solid compound in a vacuum packaging.

13. The method according to claim 1 wherein the food product is a food supplements, vitamin supplements, therapeutic compositions, or cosmetic compositions.
